(19) United States
(12) Patent Application Publication
(10) Pub. No.: US 2013/0103001 A1
(43) Pub. Date: Apr. 25, 2013

(54) Side-Loading Torque Device

(71) Applicant: IMDS Corporation, Logan, UT (US)

(72) Inventors: Moutaa BenMaamer, Ft. Myers, FL (US); Brad Anthony Niese, Chandler, AZ (US)

(73) Assignee: IMDS CORPORATION, Logan, UT (US)

(21) Appl. No.: 13/653,770

(22) Filed: Oct. 17, 2012

Related U.S. Application Data
(60) Provisional application No. 61/548,794, filed on Oct. 19, 2011.

(51) Int. Cl. A61M 25/09

(52) U.S. Cl. 604/528

(57) ABSTRACT

A surgical instrument for grasping and controlling a guide wire includes a male member having a longitudinal slit in communication with an inner hollow bore and a female member having a longitudinal slit in communication with an inner chamber. The male member is rotatably engageable within at least a portion of the female member. A method of threading a guide wire into a surgical instrument includes engaging a male member within a female member, rotating the male member relative to the female member until a longitudinal slit of the male member is aligned with a longitudinal slit of the female member; inserting a guide wire through the slits and into an interior of the male member and female member; and rotating the male member relative to the female member until the slits of the male member and female member are not aligned, trapping the guide wire within the surgical instrument.

### CROSS-REFERENCE TO RELATED APPLICATIONS

This application is a non-provisional of:

pending U.S. Provisional Patent Application No. 61/548,794, filed Oct. 19, 2011, which carries Applicant's docket No. MLI-101 PROV, and is entitled SIDE-LOADING TORQUE DEVICE.

The above-identified document is incorporated herein by reference.

### FIELD OF THE INVENTION

This invention relates to guidewires used in diagnostic and interventional medical procedures and, more particularly, to a torque device including a body disposed on a guidewire used to position the guidewire within a body for an endovascular procedure.

### BACKGROUND OF THE INVENTION

Guidewires (also known as wire guides) have been used in percutaneous entry procedures for diagnostic X-Ray studies and interventional procedures since the 1950's when the idea of percutaneous, guidewired entry into the vasculature was conceived. Typically, guidewires are inserted percutaneously into a body vessel, such as a vein or artery, and advanced or manipulated within the body vessel until reaching a desired location. A catheter (or other insertable device) is then positioned over the guidewire, inserted percutaneously into the body vessel, and advanced along the guidewire to a desired location to perform a desired treatment, diagnosis, investigation, or medical intervention.

Therefore, guidewires typically have particular characteristics to improve the pushability of the guidewire within the body vessel. For example, the guidewire is preferably generally radially flexible to negotiate the potentially-winding path of the body vessel and to reduce potential damage to the body vessel walls while the guidewire is being advanced. More specifically, the guidewire preferably has a relatively high axial stiffness to improve the pushability and control of the guidewire along the body vessel. The relatively high axial stiffness reduces kinking and bending so that the guidewire will not become stuck or obstructed during the advancement thereof along the body vessel. The axial stiffness of the guidewire is preferably sufficient to prevent the guidewire from folding over itself and becoming obstructed within the body vessel when the distal tip encounters a bend or curve in the body vessel.

However, during positioning of the guidewire and advancement through the body vessel, it may be required to adjust the guidewire to advance through a desired branch of the body vessel. To advance the guidewire through the desired branch, the guidewire may need to be moved laterally or rotated. Given a distance between a distal end of the guidewire and a portion of the guidewire outside the body that may be manipulated by a doctor, the distal end of the guidewire may be difficult to advance through the desired branch. Furthermore, a length of guidewire outside of the body of the patient is as long as or longer than a length of guidewire within the patient. Therefore, guiding and/or rotating the distal end of the guidewire may be difficult. Also, once a guidewire is positioned within the body vessel in a desired location, the guidewire may be accidentally advanced or pulled from the body vessel requiring a doctor to reposition the guidewire.

It would be desirable to provide a torque device adapted to facilitate advancement and/or rotation of the guidewire through a body vessel that is able to maintain a position of the guidewire at a desired location and that may be easily removed from the guidewire.

### SUMMARY OF THE INVENTION

Concordant and congruous with the present invention, a torque device adapted to facilitate advancement and/or rotation of the guidewire through a body vessel that is able to maintain a position of the guidewire at a desired location and that may be easily removed from the guidewire has surprisingly been discovered.

According to an embodiment of the invention, a torque device comprises a cap having a first end and a second end and forming a hollow passage therethrough and a first channel extending along a longitudinal axis thereof; a body having a first end and a second end and forming a hollow passage therethrough and a second channel extending along a longitudinal axis thereof, the first end of the body received within the hollow passage of the cap; and an annular array of spaced apart protuberances formed on the first end of the body, the protuberances adapted to selectively compress when the first end of the body is selectively advanced through the hollow passage of the cap toward the first end thereof.

According to another embodiment of the invention, a surgical instrument for grasping and controlling a guide wire, comprises a male member having a longitudinal slit in communication with an inner hollow bore; and a female member having a longitudinal slit in communication with an inner chamber of the female member, wherein the male member is rotatably engageable within at least a portion of the female member.

According to another embodiment of the invention, a method of threading a guide wire into a surgical instrument comprises engaging a male member within a female member, wherein the male member comprises a longitudinal slit and wherein the female member comprises a longitudinal slit; rotating the male member counterclockwise relative to the female member until the longitudinal slit of the male member is substantially aligned with the longitudinal slit of the female member; inserting a guide wire through the substantially aligned longitudinal slits and into an interior of the male member and female member; and rotating the male member clockwise relative to the female member until the longitudinal slits of the male member and female member are not substantially aligned, thereby trapping the guide wire within the surgical instrument.

### BRIEF DESCRIPTION OF THE DRAWINGS

The above, as well as other objects and advantages of the invention, will become readily manifest to those skilled in the art from reading the following detailed description when considered in light of the attached drawings. It is appreciated that these drawings depict only typical embodiments of the invention and are therefore not to be considered limiting of its scope.

FIG. 1 is a side view of a back loading torque device known in the prior art with male and female members disconnected and a wire guide threaded through both of the female and male members;

FIG. 2 is a side view of the back loading torque device of FIG. 1 with the male and female members connected to each other;

FIG. 3 is a side view of a different embodiment of a back loading torque device known in the prior art with the male and female members translated distal from each other;

FIG. 4 is a side view of the back loading torque device of FIG. 3 with the male and female members in closer proximity to each other;

FIG. 5 is a side view of a side loading torque device according to an embodiment of the invention with the male and female members disconnected and a guide wire disposed there through;

FIG. 6 is a side view of the torque device of FIG. 5 with the male and female members connected to each other and their longitudinal slits in alignment with a guide wire disposed therein;

FIG. 7 is a side view of the torque device of FIG. 6 with the male and female members connected to each other with their respective longitudinal slits not in alignment with each other and a guide wire disposed there through;

FIG. 8 is a cross-sectional view of the torque device of FIG. 7;

FIG. 9 is an end elevational view of the torque device of FIG. 7 illustrating a longitudinal slit with opposing parallel sides;

FIG. 10 is an end elevational view of a torque device according to another embodiment of the invention illustrating a longitudinal slit with opposing sides which converge towards each other from the inside of the torque device toward the outside the torque device;

FIG. 11 is an end elevation view of a torque device according to another embodiment of the invention illustrating a longitudinal slit with opposing sides which diverge towards each other from the inside of the torque device toward the outside of the torque device;

FIG. 12 is an end elevation view of a torque device according to another embodiment of the invention illustrating a longitudinal slit which is offset from the center longitudinal axis and has a divergent portion closer to the outer edge of the torque device;

FIG. 13 is an isometric view of a side loading torque device with male and female members connected to each other having their longitudinal slits in alignment with their stop members engaged;

FIG. 14 is an isometric view of the side loading torque device of FIG. 13 with a guide wire about to be threaded into the longitudinal slits of the torque device;

FIG. 15 is an isometric view of the side loading torque device of FIG. 14 with the guide wire threaded into the longitudinal slits of the torque device;

FIG. 16 is an isometric view of the side loading torque device of FIG. 15 with the guide wire threaded inside the torque device and the male member rotated clockwise such that the longitudinal slits no longer are in alignment, thereby locking the guide wire within the torque device;

FIG. 17 is a top view of the torque device of FIG. 16 showing a cross-sectional line 18A-18B;

FIG. 18 is a cross-sectional side view of the torque device of FIG. 17 taken along the line 18A-18B in FIG. 17;

FIG. 19 is an isometric view of the torque device of FIG. 18 with stop members connected to the male and female members and engaged with each other aligning the longitudinal slits;

FIG. 20 is an isometric view of the torque device of FIG. 19 with the stop members disengaged with each other and the longitudinal slits out of alignment;

FIG. 21 is an isometric view of the male member of FIG. 20 showing the collet locking mechanism and male stop member; and

FIG. 22 is an isometric view of the female member of FIG. 21 showing the inside threaded portion of the female member, the longitudinal slit, and the female stop member.

### DETAILED DESCRIPTION OF THE PREFERRED EMBODIMENTS

The following detailed description and appended drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

In this specification, a standard system of three mutually perpendicular reference planes is employed. A sagittal plane divides a body into bilaterally symmetric right and left portions. A coronal plane divides a body into anterior and posterior portions. A transverse plane divides a body into superior and inferior portions.

Guide wires are used in most catheter-based procedures. The distal end of a guide wire typically has an angled tip, which can be oriented to help steer the guide wire through curves and junctions of the vasculature or vessels of a patient. The orientation of the angled tip is achieved by torqueing the guide wire so that it rotates about its axis. However, since the guide wire has a small diameter and typically a smooth surface, it is difficult to torque with an operator's fingers. Torqueing requires the aid of a larger diameter torque device, which is attached to the guide wire. A torque device is used to provide a “handle” whereby the surgeon can have maximum control over the positioning and orientation of the guide wire.

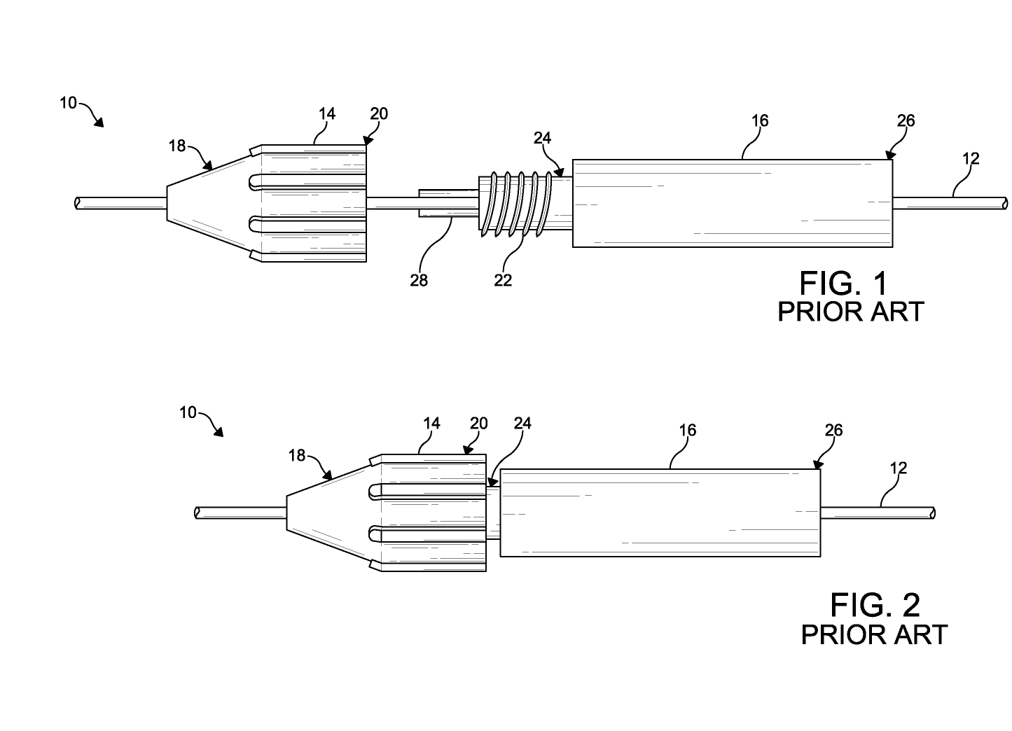

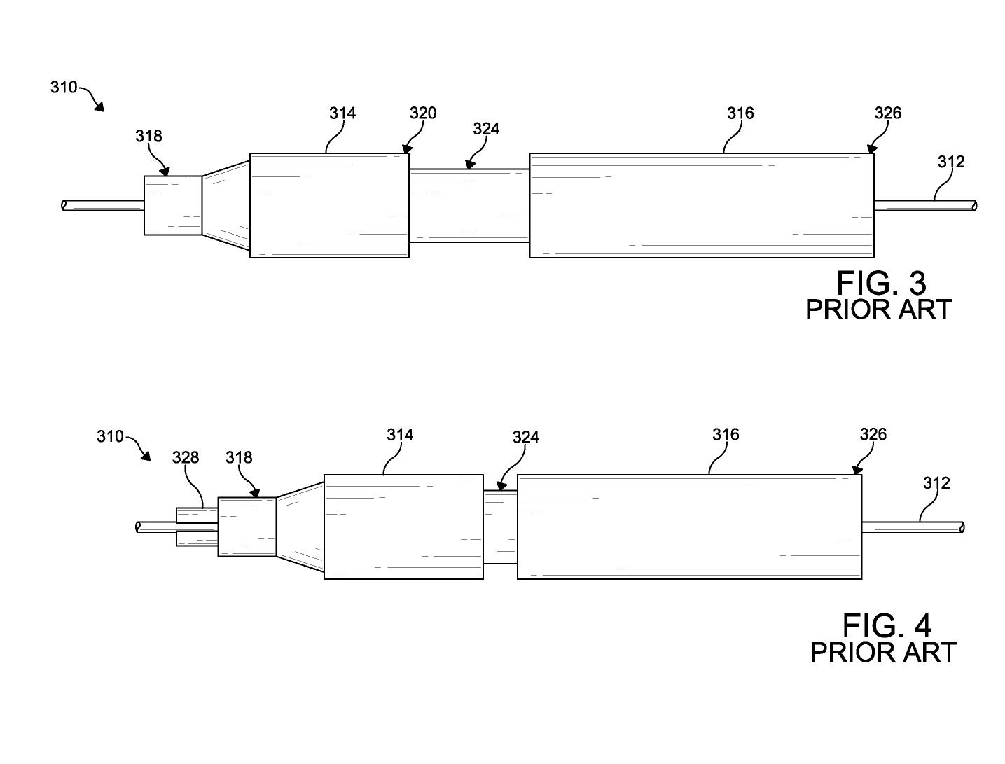

One of the disadvantages of guide wire torque devices known in the art, such as those shown in FIGS. 1-4, is that they must generally be “back loaded” over the proximal end of a guide wire, costing the surgeon precious time and attention in order to thread the torque device over the guide wire. In addition, to remove these types of devices to dispose tools or implements required to perform a procedure, such as catheters, and the like, the device must be slidingly removed from the guidewire which may have a great length. During removal of this device, the guidewire may be moved or accidentally pulled out of the patient, thereby requiring the guidewire positioning procedure to be repeated.

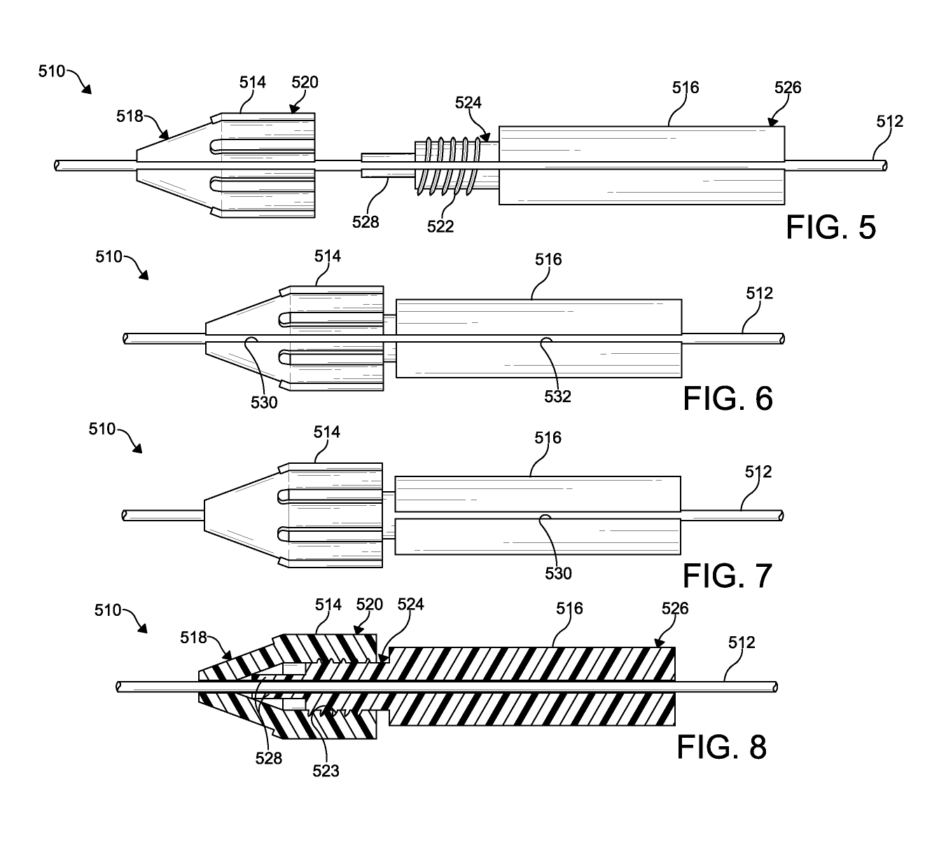

One way to alleviate this problem is to create a slit or longitudinal aperture in both the male 532 and female 530 parts of the torque device as shown in FIGS. 5-12. This allows a surgeon to “side load” the guide wire into the torque device, obviating the need to load the torque device from the proximal end of the guide wire.

FIGS. 5-12 illustrate a torque device 510 according to an embodiment of the invention. The torque device 510 is disposed on a guidewire 512 for advancement within a body vessel (not shown), such as a vein or an artery, for example. The torque device 510 includes a cap 514 and a cooperating body 516. As shown, the cap 514 and the body 516 are formed from a plastic, but the cap 514 and the body 516 may be formed from a metal, such as a stainless steel, for example, as desired.

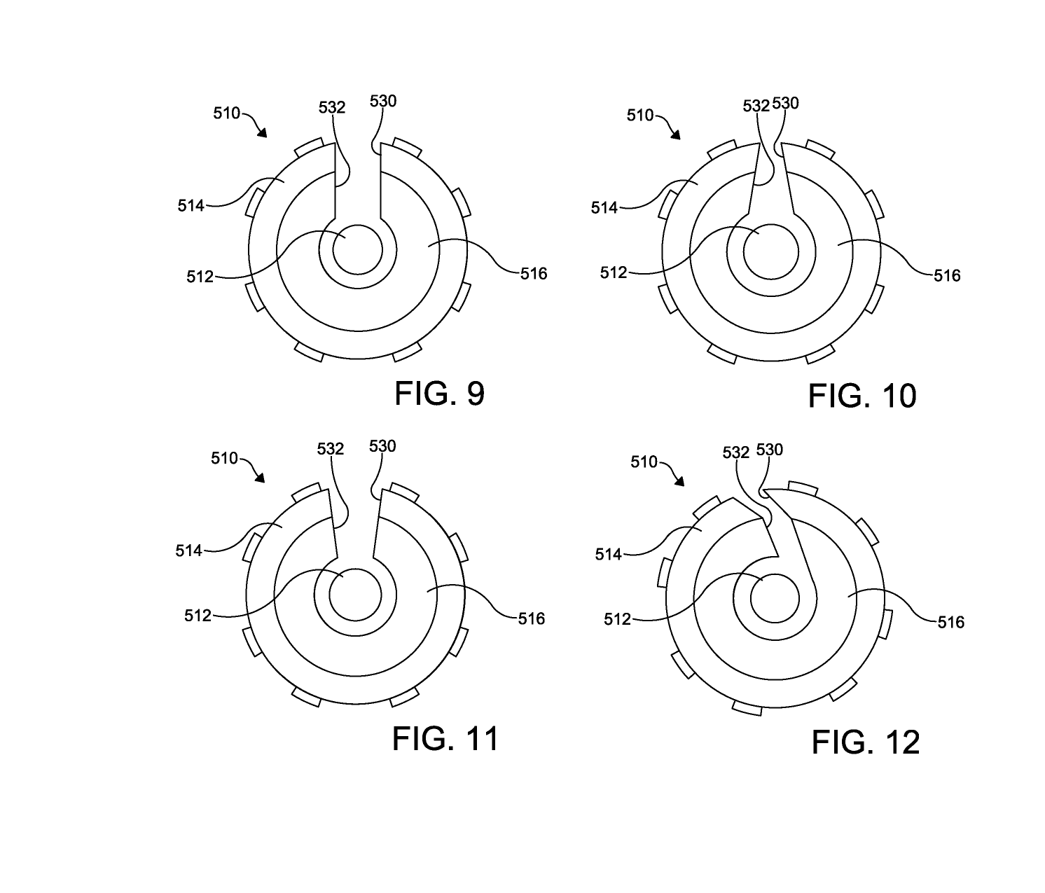

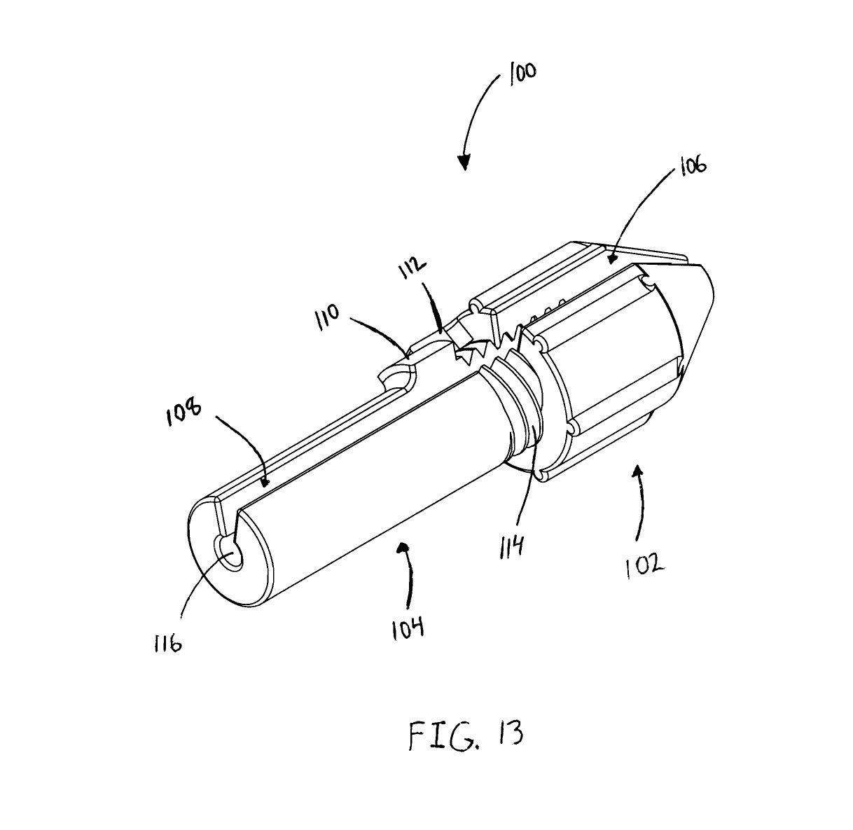

The cap 514 includes a first end 518 and a second end 520. A hollow passage extends between the first end 518 and the second end 520. A channel 530 providing communication with the passage is formed along a longitudinal axis of the cap 514. The channel 530 may form a substantially linear aperture, as shown in FIG. 9; an aperture tapering away from the guidewire 512 to facilitate removal of the guidewire 512 therefrom, as shown in FIG. 10; an aperture tapering toward guidewire 512 to militate against removal of the guidewire 512 therefrom, as shown in FIG. 11; or the aperture may formed at an angle with respect to plane tangential to the cap 514 to militate against removal of the guidewire 512 therefrom, as shown in FIG. 12. An aperture formed in the first end 518 and an aperture formed in the second end 520 provide access to the passage and facilitate advancement of the guidewire 512 through the cap 514. The first end 518 has a conical shape, and a portion of the passage formed in the first end 518 is narrower than a portion of the passage formed through the second end 520. As shown in FIG. 8, threads 523 formed adjacent the second end 520 on an interior of the cap 514 cooperate with threads 522 on a first end 524 of the body 516.

The body 516 includes the first end 524 and a second end 526. A hollow passage extends between the first end 524 and the second end 526. A channel 532 providing communication with the passage is formed along a longitudinal axis of the body 516. The channel 532 may form a substantially linear aperture, as shown in FIG. 9; an aperture tapering away from the guidewire 512 to facilitate removal of the guidewire 512 therefrom, as shown in FIG. 10; an aperture tapering toward guidewire 512 to militate against removal of the guidewire 512 therefrom, as shown in FIG. 11; or the aperture may formed at an angle with respect to plane tangential to the body 516 to militate against removal of the guidewire 512 therefrom, as shown in FIG. 12. An aperture formed in the first end 524 and an aperture formed in the second end 526 provide access to the passage and facilitate advancement of the guidewire 512 through body 516. The first end 524 includes an annular array of spaced apart protuberances 528 cooperating to form a portion of the passage through the first end 524. The protuberances 528 are flexible and adapted to be selectively compressed around the guidewire 512 to militate against advancement thereof through the body 516. The threads 522 of the body 516 are formed intermediate the protuberances 528 and the second end 526. It is understood that an annular channel (not shown) adapted to receive and cooperate with a detent (not shown) formed on an interior of the cap 514 may be formed between the protuberances 528 and the threads 522. The annular channel and the detent cooperate to militate against removal of the first end 524 of the body 516 from the cap 514 once the torque device 510 is assembled.

In use, the cap 514 and the body 516 are assembled to form the torque device 510 with the first end 524 of the cap 514 disposed within the passage of the cap 514 adjacent the second end 520 thereof. The cap 514 and the body 516 may be assembled by clicking the detent into the annular channel, or by rotating the body 516 to engage the threads 522 thereof with the threads 523 formed on the interior of the cap 514. The passages of the cap 514 and the body 516 cooperate to form a unitary passage through which the guidewire 512 is fed when the torque device 510 is assembled. It is understood that the cap 514 and the body 516 may include indicia thereon to indicate when the channels 530, 532 are properly aligned for insertion of the guidewire as in FIG. 6, and when the channels 530, 532 are appropriately misaligned for use of the torque device 510, as in FIG. 7.

The guidewire 512 is disposed through the aperture formed in the second end 526 of the body 516, through the passage formed in the body 516, through the passage formed in the cap 514, and through the aperture formed in the first end 518 of the cap 514. Alternatively, the grooves 530, 532 of the assembled torque device 510 may be aligned, as shown in FIG. 5 so that the guidewire 512 may be disposed therethrough. An end of the guidewire 512 is then disposed through a puncture in the skin of a patient and into a body vessel (not shown). Using x-ray pictures as a guide, the guidewire 512 is advanced through the body vessel. When a branch or bend in the body vessel is reached, the body 516 is rotated and threaded into the cap 514 to advance the first end 524 toward the first end 518 of the cap 514. As the first ends 524, 518 converge, the protuberances 528 advance into the narrowing passage of the conical portion of the cap 514 causing the protuberances 528 to compress around the guidewire 512. Once the protuberances 528 are compressed around the guidewire 512, the torque device 510 is in a closed position and advancement of the guidewire 512 through the torque device 510 is militated against. In the closed position, the groove 530 is offset from the groove 532, as shown in FIG. 7, to militate against removal of the guidewire 512 therefrom. The torque device 510 may be gripped by the doctor to rotate and/or laterally move the guidewire 512 disposed within the body vessel of the patient, or the guidewire 512 disposed between the puncture and the torque device 510 may be gripped by the doctor to rotate and/or laterally move the guidewire 512 disposed within the body vessel of the patient.

The body 516 may then be unscrewed from the cap 514 to allow the guidewire 512 to be fed therethrough. Once the end of the guidewire 512 is in a desired location within the body vessel, the torque device 510 is returned to the closed position with the torque device 510 adjacent the puncture in the patient. If the guidewire 512 is inadvertently withdrawn from the patient, the guidewire 512 and the torque device 510 may be re-advanced until the torque device 510 is again adjacent the puncture. Once the guidewire 512 is in the desired location and advancement through the body vessel is complete, the torque device 510 may be opened and slidably removed from the guidewire 512. Once the guidewire 512 is in place, the doctor can proceed with the endovascular procedure. To remove the torque device 510 to dispose tools or implements required to perform the procedure, such as catheters, and the like on the guide wire 512, the cap 514 and the body 516 are positioned to align the channels 530, 532 to form a unitary channel that extends the length of the torque device 510 and the torque device 510 may be removed laterally from the guidewire 512 without having to slidably remove the torque device 510 longitudinally from the guidewire 512 which may be very long.

FIGS. 9-12 show end elevational views of the torque devices of FIGS. 5-8 and illustrate how the longitudinal slits in the male and female members create an opening through which a guide wire may be inserted in a side loading fashion into the interior of the torque device. The surgeon can use one or both hands to grasp the side-loading endovascular torque device and rotate the male member in relation to the female member such that the slits of both the male member and the female member align. The surgeon may then insert the guide wire through the side slits, and rotate the male and female members relative to each other such that the side slits no longer align causing the guide wire to be retained within the torque device. This reduces the time needed for the surgeon to thread the guide wire into the torque device. However, the surgeon still needs to look at the orientation of the slits in both the male and female members in order to verify that both slits are in alignment before inserting the guide wire into the torque device.

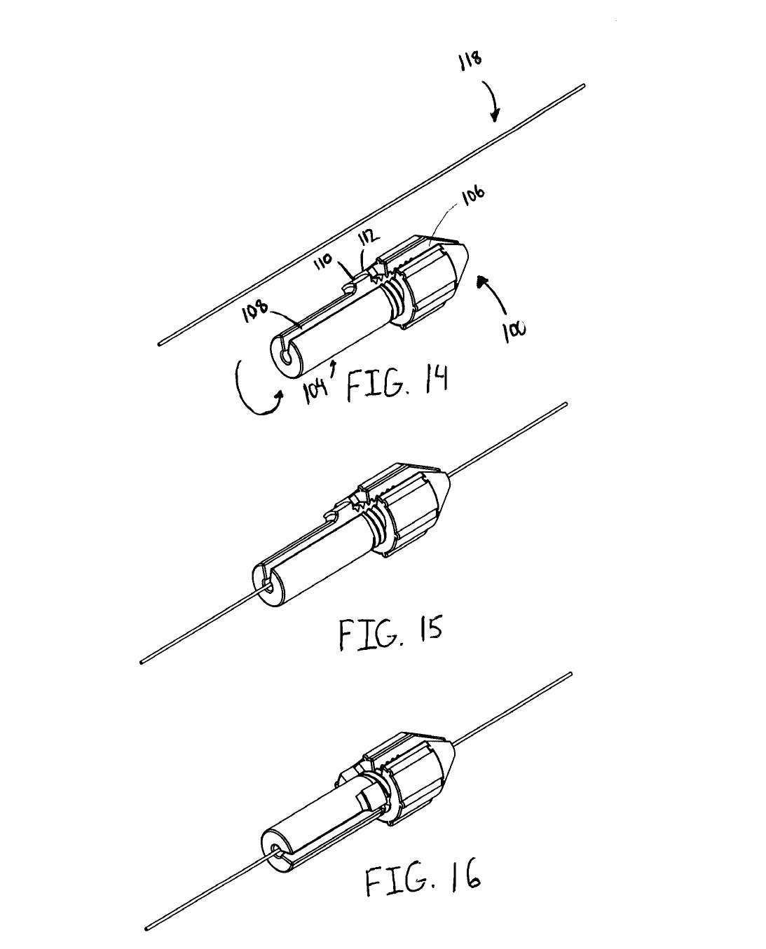

Another embodiment to further reduce the time and effort required by the surgeon to thread the guide wire into the side loading torque device is seen in FIGS. 13-22. FIG. 13 shows a perspective view of a side loading torque device having male member 104 engaged within female member 102. Male member 104 may have threads 114 to threadably engage within female member 102 which may also have complementary threads disposed with in female member 102 and configured to receive male member 104. Male member 104 has a male longitudinal slit or aperture 108 which spans the length of male member 104 Likewise, female member 102 has a female longitudinal slit or aperture 106 which spans the length of female member 102. Male longitudinal slit 108 and female longitudinal slit 106 can be better seen in FIGS. 21 and 22, wherein the male member 104 is disconnected from the female member 102.

Referring back to FIG. 13, male member 104 may also have a hollow inner bore 116 spanning the length of male member 104 which may be configured to receive a guide wire within the hollow inner bore 116. Male longitudinal slit 108 may be in communication with, or configured to allow access to, the hollow inner bore 116 such that a guide wire (not shown) can pass through male longitudinal slit 108 and into hollow inner bore 116 in a “side loading” fashion. Male member 104 may also have a male stop member 110 protruding from a surface of male member 104 Likewise, female member 106 may also include a female stop member 112 protruding from a surface of female member 102. Male stop member 110 and female stop member 112 may be shaped so as to engage each other in such a fashion as to prevent rotation of male member 104 within female member 102 when male longitudinal slit 108 is aligned with female longitudinal slit 106.

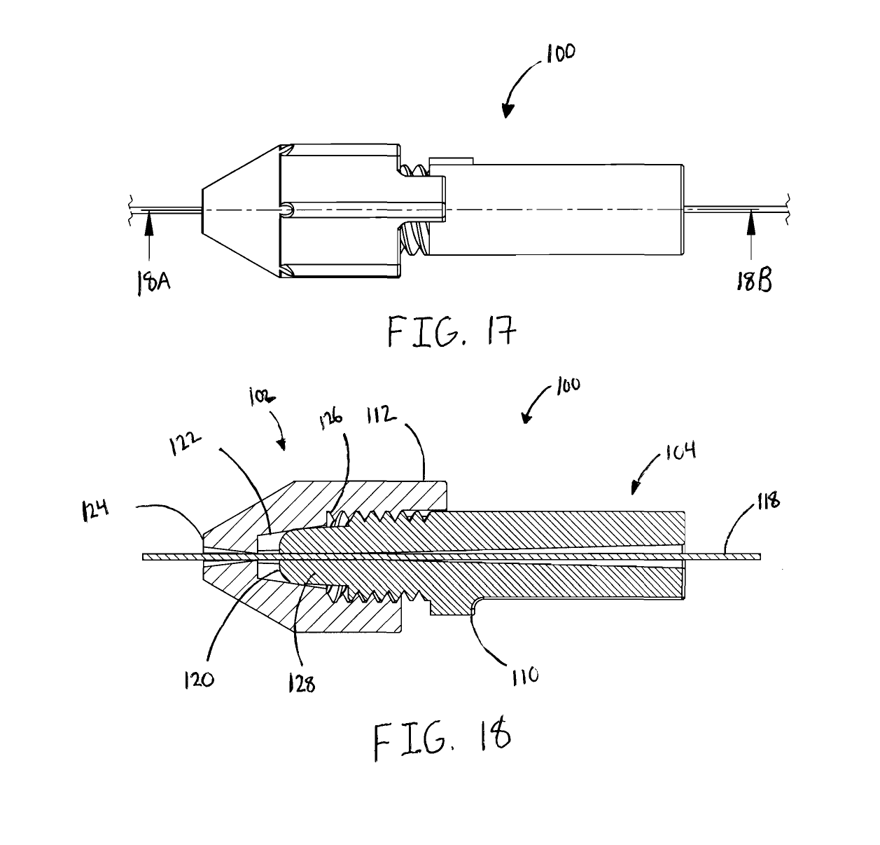

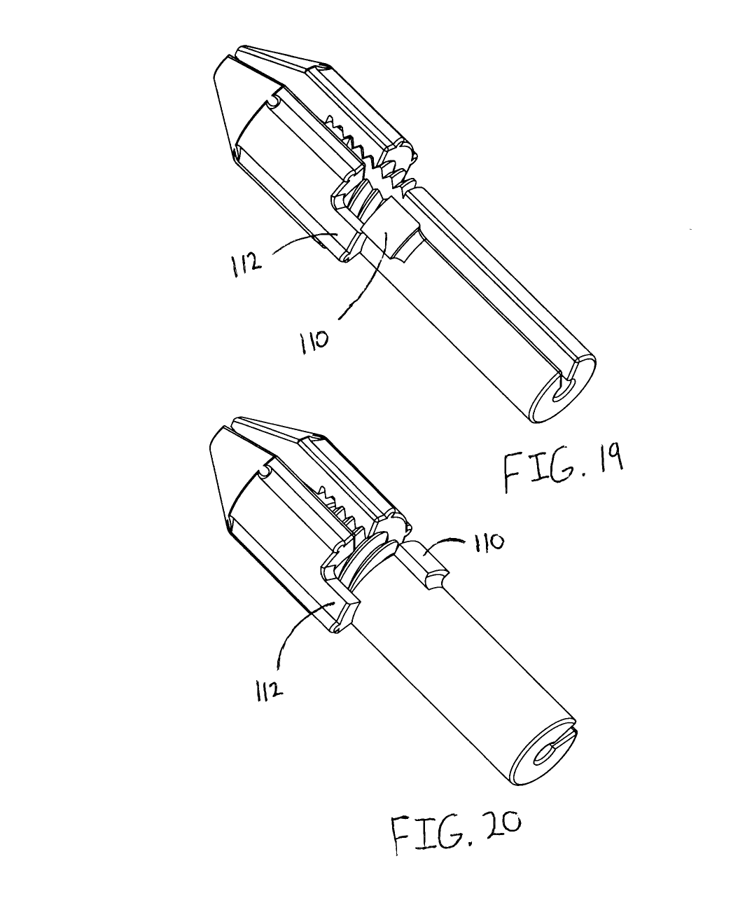

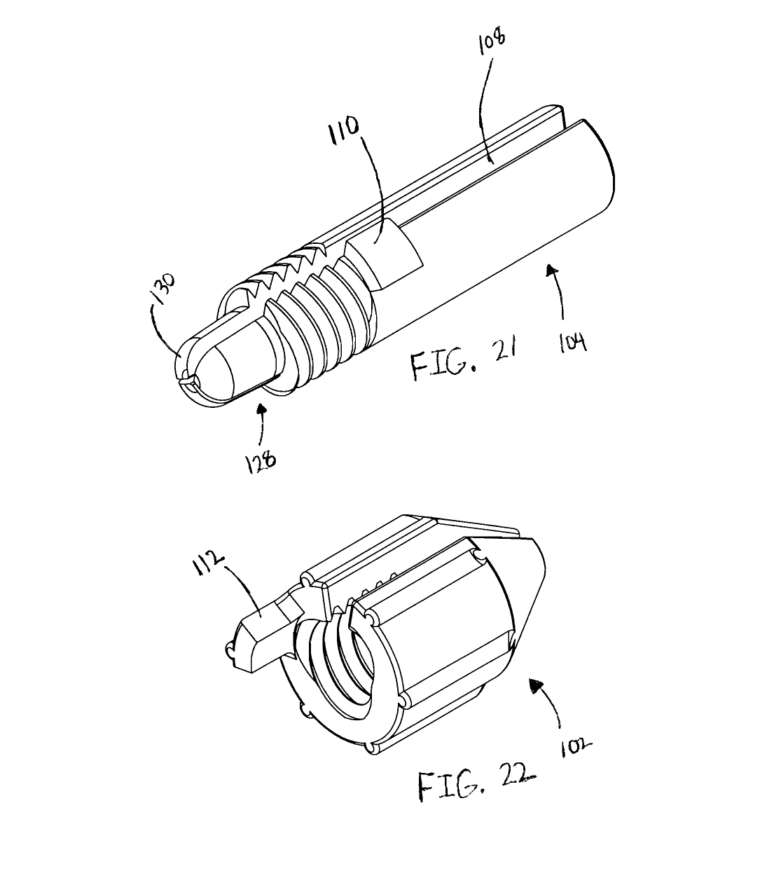

FIGS. 14-16 illustrate how a guide wire 118 is side-loaded it into a torque device 100 according to the present disclosure. In operation, a surgeon can rotate male member 104 counterclockwise until male stop member 110 engages with female stop member 112 preventing the surgeon from further rotation in the counterclockwise direction. At the point which male stop member 110 engages with female stop number 112, male longitudinal slit 108 is substantially in alignment with female longitudinal slit 106 and ready to receive the guide wire 118, as can be seen in FIG. 14. In this orientation, the slits of the collet style locking mechanism 128 (see FIG. 21) at the distal end of male member 104 may also be substantially aligned with the longitudinal slits of the male and female members. Furthermore, the collet style locking mechanism 128 may also be substantially uncompressed, thereby not exerting a substantial amount of frictional force upon a guide wire to be inserted therein. The surgeon can then side-load guide wire 118 into the torque device 104, as is shown in FIG. 15. The surgeon then may rotate male member 104 in the clockwise direction such that male longitudinal slit 108 and female longitudinal slit 106 are no longer in alignment, thereby retaining guide wire 118 disposed within torque device 100 as can be seen in FIG. 16. Furthermore, with sufficient clockwise rotation the surgeon can actuate the collet locking mechanism 128 (shown in more detail in FIGS. 18 and 21 and discussed in more detail below) to produce sufficient frictional force to grasp the guide wire 118 thereby locking the guide wire 118 relative to the torque device 100. FIGS. 19 and 20 illustrate another isometric view of the torque device 100 according to the present disclosure with the stop members engaged in FIG. 19 and disengaged in FIG. 20.

FIGS. 17 and 18 illustrate a cross-sectional view of torque device 100 with FIG. 18 showing the cross-sectional view of the torque device of FIG. 17 taken along the line 18A-18B in FIG. 17. FIG. 18 shows the distal end 120 of male member 104 disposed with in female member 102. The distal end 120 of the male member 104 may comprise a compression device such as a collet locking mechanism 128 (shown in more detail in FIG. 21). The interior within female member 102, intermediate the tip 124 of female member 102 and the threaded portion 126 of female member 102, may comprise a chamber 122 configured to receive the distal end 120 of male member 104. The chamber 122 may have a conical shape with a larger interior space volume at the end of chamber 122, closest to the threaded portion 126, and a smaller interior space volume at the end of chamber 122, closest to the tip 124 of female member 102. As the surgeon rotates male member 104 clockwise, male member 104 enters further and further into female member 102. As the collet locking mechanism 128 enters further and further into chamber 122, the collet 128 is compressed into a smaller and smaller space causing the flanges 130 (see FIG. 21) to compress together and frictionally engage the guide wire 118. With enough compression, the collet locking mechanism 128 exerts sufficient frictional force upon the guide wire 118 such that the guide wire 118 is “locked” relative to torque device 100, preventing the guide wire 118 from sliding freely within the hollow inner bore 116 of torque device 100.

Either or both of the stop members 110, 112 may be deflectable to permit the stop members to rotate past each other upon initial assembly of the torque device 100. Either or both of the stop members 110, 112 may be attachable to the corresponding member 102, 104 after initial assembly of the members 102 and 104.

It should be understood that the present system, kits, apparatuses, and methods are not intended to be limited to the particular forms disclosed. Rather, they are to cover all modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The present invention may be embodied in other specific forms without departing from its spirit or essential characteristics. It is appreciated that various features of the above-described examples can be mixed and matched to form a variety of other alternatives. For example, indicia and/or stop members may be included on any of the embodiments. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A surgical instrument for grasping and controlling a guide wire, comprising
a male member having a longitudinal slit in communication with an inner hollow bore; and
a female member having a longitudinal slit in communication with an inner chamber of the female member, wherein the male member is rotatably engageable within at least a portion of the female member.

2. The surgical instrument of claim 1, wherein the male member further comprises a stop member engageable with at least a portion of the female member.

3. The surgical instrument of claim 2, wherein the female member further comprises a stop member engageable with the stop member of the male member.

4. The surgical instrument of claim 3, wherein the longitudinal slits of both the male and female members are substantially aligned with each other when the male stop member is engaged with the female stop member permitting a guide wire to be threaded through the substantially aligned longitudinal slits.

5. The surgical instrument of claim 1, wherein the male member and the female member further comprise threaded portions which rotatably engage with each other.

6. The surgical instrument of claim 1, wherein an end elevational view of the surgical instrument viewed in the direction of the longitudinal axis further comprises longitudinal slits having sides parallel to each other.

7. The surgical instrument of claim 1, wherein an end elevational view of the surgical instrument viewed in the direction of the longitudinal axis further comprises longitudinal slits having sides which converge toward each other in a direction moving from the center of the longitudinal axis toward the outer edge of the surgical instrument.

8. The surgical instrument of claim 1, wherein an end elevational view of the surgical instrument viewed in the direction of the longitudinal axis further comprises longitudinal slits having sides which diverge away from each other in a direction moving from the center of the longitudinal axis toward the outer edge of the surgical instrument.

9. The surgical instrument of claim 1, wherein an end elevational view of the surgical instrument viewed in the direction of the longitudinal axis further comprises longitudinal slits having sides offset from the center longitudinal axis and a divergent portion closer to the outer edge of the torque device.

10. The surgical instrument of claim 3, wherein when the longitudinal slits of both the male and female members are not substantially aligned with each other, a guide wire disposed within the surgical instrument is not permitted to pass through the longitudinal slits.

11. A method of threading a guide wire into a surgical instrument comprising:
engaging a male member within a female member, wherein the male member comprises a longitudinal slit and wherein the female member comprises a longitudinal slit;
rotating the male member counterclockwise relative to the female member until the longitudinal slit of the male member is substantially aligned with the longitudinal slit of the female member;
inserting a guide wire through the substantially aligned longitudinal slits and into an interior of the male member and female member; and
rotating the male member clockwise relative to the female member until the longitudinal slits of the male member and female member are not substantially aligned, thereby trapping the guide wire within the surgical instrument.

12. The method of claim 11, further comprising rotating the male member clockwise relative to the female member to engage a collet locking mechanism to exert compression force on the guide wire to substantially hold the guide wire from translating within the surgical instrument.

13. The method of claim 11, wherein rotating the male member counterclockwise relative to the female member until the longitudinal slit of the male member is substantially aligned with the longitudinal slit of the female member further comprises automatically stopping the counterclockwise rotation with male and female stop members.
